(19) United States
(12) Patent Application Publication
(10) Pub. No.: US 2003/0187076 A1
(43) Pub. Date: Oct. 2, 2003

(54) Non-steroidal analogs of 2-methoxyestradiol

(76) Inventors: Gregory E. Agoston, Germantown, MD (US); Jamshed H. Shah, Brookeville, MD (US); Kimberly A. Hunsucker, Alpharetta, GA (US); Anthony M. Treston, Rockville, MD (US); Victor S. Pribluda, Silver Spring, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/354,921

(22) Filed: Jan. 30, 2003

Related U.S. Application Data
(60) Provisional application No. 60/354,046, filed on Jan. 30, 2002.

(51) Int. Cl. A61K 31/075; C07C 43/02; C07C 43/20

(52) U.S. Cl. 514/718; 568/633

(57) ABSTRACT

Compositions and methods for treating mammalian disease characterized by undesirable angiogenesis and proliferative activity by administering non-steroidal derivatives of 2-methoxyestradiol of the general formula:

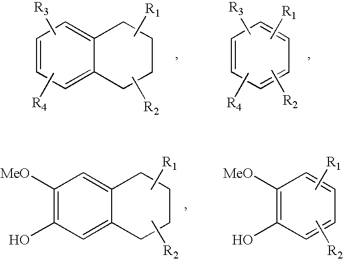

wherein the variables are defined in the specification.

### PRIOR RELATED U.S. APPLICATION DATA

This application claims priority to U.S. Provisional Patent Application Serial No. 60/354,046 filed Jan. 30, 2002, the entirety of which is incorporated herein by reference.

### FIELD OF THE INVENTION

The present invention relates to treating disease states characterized by abnormal cell mitosis and to treating disease states characterized by abnormal angiogenesis and to treating disease states characterized by a combination of these events. More particularly, the present invention relates to non-steroidal analogs of 2-methoxyestradiol (2ME₂) and their effect on diseases characterized by abnormal cell mitosis and/or abnormal angiogenesis and/or abnormal proliferative activity, including their effect on tumors.

### BACKGROUND OF THE INVENTION

Angiogenesis is the generation of new blood vessels into a tissue or organ. Under normal physiological conditions, humans and animals undergo angiogenesis only in very specific, restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogenesis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in many disease states, tumor metastases, and abnormal growth or proliferation by endothelial cells. The diverse pathological disease states in which unregulated angiogenesis is present have been grouped together as angiogenic-dependent or angiogenic-associated diseases.

One example of a disease mediated by angiogenesis and proliferative activity is ocular neovascular disease. This disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, and pterygium keratitis sicca. Other diseases associated with undesirable angiogenesis include Sjögren's syndrome, acne rosacea, phylectenulosis, syphilis, Mycobacteria infections, lipid degeneration, chemical burns, bacterial ulcers, fungal ulcers, Herpes simplex infection, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's syndrome, sarcoidosis, scleritis, Stevens-Johnson's disease, pemphigoid, and radial keratotomy.

Diseases associated with retinal/choroidal neovascularization and endothelial proliferative activity include, but are not limited to, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacteria infections, lyme's disease, systemic lupus erythematosis, retinopathy of prematurity, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other eye-related diseases include, but are not limited to, diseases associated with rubeosis (neovascularization of the angle) and diseases caused by the abnormal proliferation of fibrovascular or fibrous tissue, including all forms of prolific vitreoretinopathy.

Another angiogenesis and proliferative activity-associated disease is rheumatoid arthritis. The blood vessels in the synovial lining of the joints undergo angiogenesis. In addition to forming new vascular networks, the endothelial cells release factors and reactive oxygen species that lead to pannus growth and cartilage destruction. Angiogenesis may also play a role in osteoarthritis. The activation of the chondrocytes by angiogenic-related factors contributes to the destruction of the joint. At a later stage, the angiogenic factors promote new bone growth. Therapeutic intervention that prevents the bone destruction could halt the progress of the disease and provide relief for persons suffering with arthritis.

Chronic inflammation may also involve pathological angiogenesis and proliferative activity. Such diseases as ulcerative colitis and Crohn's disease show histological changes with the ingrowth of new blood vessels and the inflamed tissues. Bartonelosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

The hypothesis that tumor growth is angiogenesis-dependent was first proposed in 1971. (Folkman, New Eng. J. Med., 285:1182-86 (1971)). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:

(1) The growth rate of tumors implanted in subcutaneous transparent chambers in mice is slow and linear before neovascularization, and rapid and nearly exponential after neovascularization. (Algire, et al., J. Nat. Cancer Inst., 6:73-85 (1945)).

(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, 164:491-502 (1966)).

(3) Tumor growth in the avascular cornea proceeds slowly and at a linear rate, but switches to exponential growth after neovascularization. (Gimbrone, Jr., et al., J. Nat. Cancer Inst., 52:421-27 (1974)).

(4) Tumors suspended in the aqueous fluid of the anterior chamber of the rabbit eye remain viable, avascular, and limited in size to <1 mm³. Once they are implanted on the iris vascular bed, they become neovascularized and grow rapidly, reaching 16,000 times their original volume within 2 weeks. (Gimbrone, Jr., et al., J. Exp. Med., 136:261-76).

(5) When tumors are implanted on the chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, 35:347-56 (1977)).

(6) Vascular casts of metastases in the rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, 68:334-40 (1970)).

(7) In transgenic mice which develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, 339:58-61 (1989)).

(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, 362:841-44 (1993)).

(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 51:6180-84 (1991)).

(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumors cells in vitro. (Gross, et al., Proc. Am. Assoc. Cancer Res., 31:79 (1990)).

(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, 48:555-57 (1990)). There is also indirect clinical evidence that tumor growth is angiogenesis dependent.

(12) Human retinoblastomas that are metastatic to the vitreous develop into avascular spheroids which are restricted to less than 1 mm³ despite the fact that they are viable and incorporate ³H-thymidine (when removed from an enucleated eye and analyzed in vitro).

(13) Carcinoma of the ovary metastasizes to the peritoneal membrane as tiny avascular white seeds (1-3 mm³). These implants rarely grow larger until one or more of them becomes neovascularized.

(14) Intensity of neovascularization in breast cancer (Weidner, et al., New Eng. J. Med., 324:1-8 (1991); Weidner, et al., J. Nat. Cancer Inst., 84:1875-87 (1992)) and in prostate cancer (Weidner, et al., Am. J. Pathol., 143(2):401-09 (1993)) correlates highly with risk of future metastasis.

(15) Metastasis from human cutaneous melanoma is rare prior to neovascularization. The onset of neovascularization leads to increased thickness of the lesion and an increased risk of metastasis. (Srivastava, et al., Am. J. Pathol., 133:419-23 (1988)).

(16) In bladder cancer, the urinary level of an angiogenic protein, bFGF, is a more sensitive indicator of status and extent of disease than is cytology. (Nguyen, et al., J. Nat. Cancer Inst., 85:241-42 (1993)).

Thus, it is clear that angiogenesis and endothelial cell proliferation play a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis and endothelium proliferation have been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed to that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly-formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

What is needed, therefore, is a composition and method which can inhibit angiogenesis. What is also needed is a composition and method which can inhibit the unwanted growth of blood vessels, especially in tumors. What is also needed is a composition and method for antiproliferative activity with respect to endothelial cell growth.

Angiogenesis is also involved in normal physiological processes, such as reproduction and wound healing. Angiogenesis is an important step in ovulation and also in implantation of the blastula after fertilization. Prevention of angiogenesis could be used to induce amenorrhea, to block ovulation, or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Several compounds have been used to inhibit angiogenesis. Taylor, et al. (Nature, 297:307 (1982)) have used protamine to inhibit angiogenesis. The toxicity of protamine limits its practical use as a therapeutic. Folkman, et al. (Science, 221:719 (1983), and U.S. Pat. Nos. 5,001,116 and 4,994,443) have disclosed the use of heparin and steroids to control angiogenesis. Steroids, such as tetrahydrocortisol, which lack glucocorticoid and mineralocorticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors, such as interferon, inhibit angiogenesis. For example, interferon alpha or human interferon beta have been shown to inhibit tumor-induced angiogenesis in mouse dermis stimulated by human neoplastic cells. Interferon beta is also a potent inhibitor of angiogenesis induced by allogeneic spleen cells. (Sidky, et al., Cancer Res., 47:5155-61 (1987)). Human recombinant interferon (alpha/A) was reported to be successfully used in the treatment of pulmonary hemangiomatosis, an angiogenesis-induced disease. (White, et al., New Eng. J. Med., 320:1197-1200 (1989)).

Other agents which have been used to inhibit angiogenesis include ascorbic acid ethers and related compounds. (Japanese Kokai Tokkyo Koho No. 58-13 (1978)). Sulfated polysaccharide DS 4152 also inhibits angiogenesis. (Japanese Kokai Tokkyo Koho No. 63-119500). Additional anti-angiogenic compounds include Angiostatin® (U.S. Pat. Nos. 5,639,725; 5,792,845; 5,885,795; 5,733,876; 5,776,704; 5,837,682; 5,861,372, and 5,854,221) and Endostatin™ (U.S. Pat. No. 5,854,205).

Another compound which has been shown to inhibit angiogenesis is thalidomide. (D'Amato, et al., Proc. Natl. Acad. Sci., 90:4082-85 (1994)). Thalidomide is a hypnosedative that has been successfully used to treat a number of angiogenesis-associated diseases, such as rheumatoid arthritis (Gutierrez-Rodriguez, Arthritis Rheum., 27 (10):1118-21 (1984); Gutierrez-Rodriguez, et al., J. Rheumatol., 16(2):158-63 (1989)), Behcet's disease (Handley, et al., Br. J. Dermatol., 127 Suppl, 40:67-8 (1992); Gunzler, Med. Hypotheses, 30(2):105-9 (1989)), graft versus host rejection (Field, et al., Nature, 211(55): 1308-10 (1966); Heney, et al., Br. J. Haematol., 78 (1):23-7 (1991)), Mycobacteria diseases (Vicente, et al., Arch. Intern. Med., 153(4):534 (1993)), Herpes simplex and Herpes zoster infections (Naafs, et al., Int. J. Dermatol., 24(2):131-4 (1985)), chronic inflammation, ulcerative colitis (Meza, et al., Drug Ther, 23 (11): 74-80, 83 (1993); Powell, et al., Br. J. Dermatol., 113 Suppl 28: 141-4 (1985)), leprosy (Barnes, et al., Infect. Immun., 60(4):1441-46 (1992)) and lupus (Burrows, BMJ, 307: 939-40 (1993)).

Although thalidomide has minimal side effects in adults, it is a potent teratogen. Thus, there are concerns regarding its use in women of child-bearing age. Although minimal, there are a number of side effects which limit the desirability of thalidomide as a treatment. One such side effect is drowsiness. In a number of therapeutic studies, the initial dosage of thalidomide had to be reduced because patients became lethargic and had difficulty functioning normally. Another side effect limiting the use of thalidomide is peripheral neuropathy, in which individuals suffer from numbness and disfunction in their extremities.

Thus, improved methods and compositions are needed that are easily administered and capable of inhibiting angiogenesis and exhibiting endothelial cell antiproliferative activity.

What is also needed are safe and effective treatments that do not create unwanted side effects.

2-Methoxyestradiol is an endogenous, steroidal metabolite of estradiol (E₂) that has potent anti-proliferative activity and induces apoptosis in a wide variety of tumor and non-tumor cell lines. When administered orally, it exhibits anti-tumor and anti-proliferative activity with little toxicity. In vitro data suggests that 2-methoxyestradiol does not engage the estrogen receptor for its anti-proliferative activity and is not estrogenic over a wide range of concentrations, as assayed by estrogen dependent MCF-7 cell proliferation. What is needed is a series of compounds that constitute analogs of 2-methoxyestradiol which are non-steroidal in structure and which will have similar biological properties to 2-methoxyestradiol and that can be used in similar applications.

### SUMMARY OF THE INVENTION

The present invention provides certain non-steroidal analogs of 2-methoxyestradiol that are effective in treating diseases characterized by abnormal mitosis and/or abnormal angiogenesis and/or abnormal proliferative activity. Specifically the present invention relates to non-steroidal analogs of 2-methoxyestradiol. Compounds within the general formulae that inhibit cell proliferation are preferred. Compounds within the general formulae that exhibit antitumor activity are also preferred. Compounds within the general formulae that inhibit angiogenesis are also preferred. Preferred compositions may also exhibit a change (increase or decrease) in estrogen receptor binding, improved absorption, transport (e.g. through blood-brain barrier and cellular membranes), biological stability, or decreased toxicity. The invention also provides compounds useful in the method, as described by the general formulae of the claims.

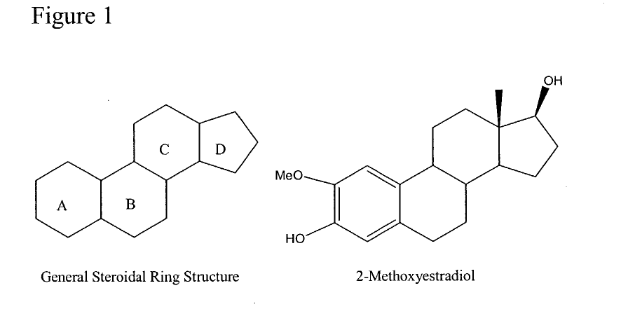

Steroids are a general class of organic molecules containing four rings (three cyclohexyl rings and one cyclopentyl ring) having the general structure in FIG. 1. The rings are generally labeled A, B, C and D. 2-Methoxyestradiol has an aromatic A ring and a methoxy substituent at position 2 and alcohols at positions 3 and 17. Structure activity relationships of estradiol analogs have been reported and have demonstrated that substituents other than methoxy (such as propyne, ethoxy and propene) at position 2 have potent in vitro antiproliferative activity (Cushman et al J. Med. Chem. 1995, 38, 2041).

A mammalian disease characterized by undesirable cell mitosis, as defined herein, includes but is not limited to excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying: rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease). Other undesired angiogenesis involves normal processes including ovulation and implantation of a blastula. Accordingly, the compositions described above can be used to block ovulation and implantation of a blastula or to block menstruation (induce amenorrhea).

It is known that 2-methoxyestradiol (2ME₂), an endogenous metabolite of estradiol with no intrinsic estrogenic activity, is a potent antiproliferative agent that induces apoptosis in a wide variety of tumor and non-tumor cell lines. When administered orally, it exhibits antitumor and antiangiogenic activity with little or no toxicity. Currently, 2ME₂ is in several phase-I and II clinical trials under the name PANZEM™.

A novel series of compounds are proposed that retain the biological activities of 2ME₂ but are expected to have varying, including reduced, metabolism. Contrary to what is observed with 2ME₂, several of these new analogs are expected to have selective in vitro antiproliferative activity for the endothelial cells over the tumor cell lines to be assessed.

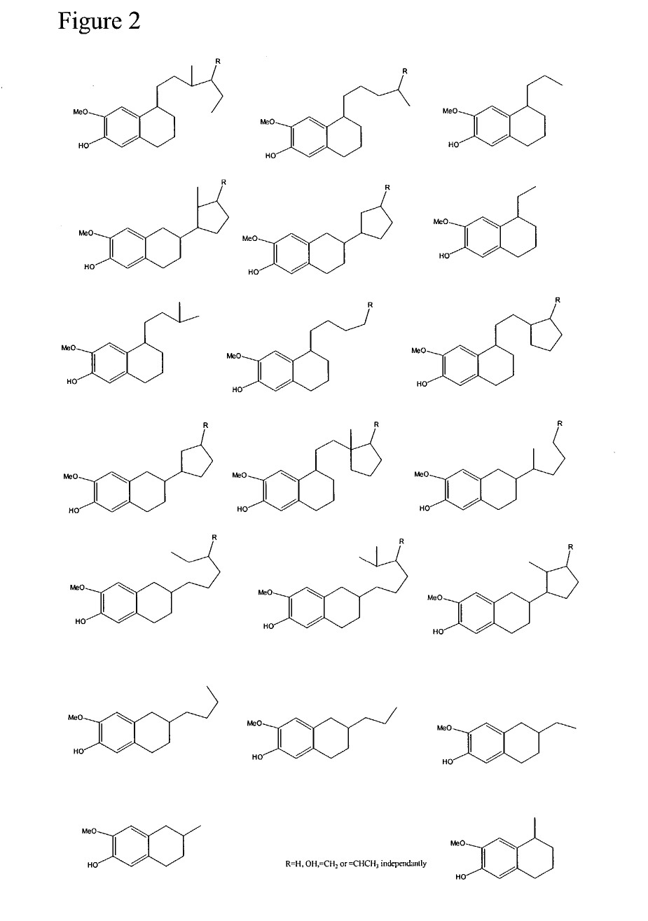

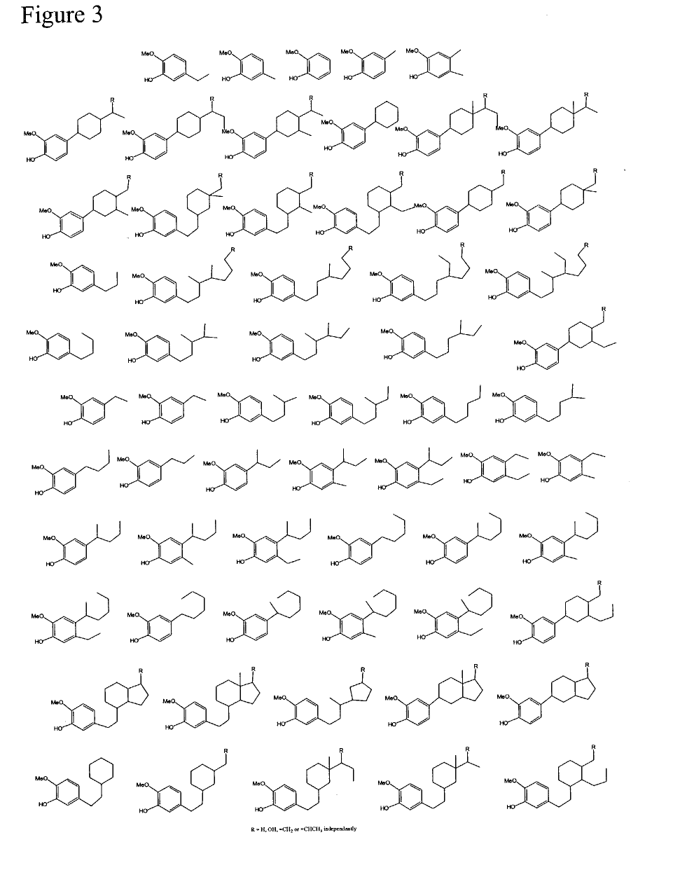

In this invention, analogs of 2-methoxyestradiol lacking portions of the four ring substructures are proposed to have similar biological activity to 2-methoxyestradiol. These analogs will have structural components of the 2-methoxyestradiol ring system (essentially they are structural fragments of 2-methoxyestradiol), but will not have the complete steroidal backbone as shown in FIG. 1. Rings that are shown in FIG. 1 as 6-member rings can also be 4, 5 or 7-member rings and may be saturated or unsaturated, and the ring shown as a five-member ring may also be a 4, 6 or 7-member ring and may be saturated or unsaturated. Examples of proposed analogs are shown in FIGS. 2 and 3, but are not limited to these compounds. Although the examples illustrated in the figures are exclusively carbon chains, it is envisioned that heteroatoms, such as O, N and S may be substituted for carbon without loss of the anti-angiogenic properties of these molecules. In all cases, it is understood by one of ordinary skill that appropriate substitutions may be made to all atoms such that they satisfy the appropriate valence. Similarly, although most of the carbon substituents are indicated as being hydrogen, some or all of these hydrogens can be replaced by more-polar moieties including but not limited to fluorines, other halides, hydroxyl, ester, amino, or alkylamine substituents which increase solubility and/or reduce metabolism and/or improve ADMET (absorption, disposition, metabolism, excretion, or toxicology) characteristics. The substituents on the unsaturated ring, which are positionally equivalent to the 2 and 3 positions of 2-methoxyestradiol and which are shown in the Figure as their preferred embodiments as methoxy and hydroxyl groups, can be replaced by groups including but not limited to halides, other alkoxy groups, propyne or other alkenes or alkynes, carboxyl or ester groups, and amines or other alkylated amino or amido groups.

Other features and advantages of the invention will be apparent from the following description of preferred embodiments thereof.

### DETAILED DESCRIPTION OF THE INVENTION

Persistent, unregulated angiogenesis occurs in a multiplicity of disease states, tumor metastasis and abnormal growth by endothelial cells and supports the pathological damage seen in these conditions. The diverse pathological disease states in which unregulated angiogenesis is present have been grouped together as angiogenic-dependent, angiogenic-associated, or angiogenic-related diseases. These diseases are a result of abnormal or undesirable cell proliferation, particularly endothelial cell proliferation.

The hypothesis that tumor growth is angiogenesis-dependent was first proposed in 1971 by Judah Folkman (N. Engl. Jour. Med. 285:1182 1186, 1971). In its simplest terms the hypothesis proposes that once tumor “take” has occurred, every increase in tumor cell population must be preceded by an increase in new capillaries converging on the tumor. Tumor “take” is currently understood to indicate a prevascular phase of tumor growth in which a population of tumor cells occupying a few cubic millimeters volume and not exceeding a few million cells, survives on existing host microvessels. Expansion of tumor volume beyond this phase requires the induction of new capillary blood vessels. For example, pulmonary micrometastases in the early prevascular phase in mice would be undetectable except by high power microscopy on histological sections. Further indirect evidence supporting the concept that tumor growth is angiogenesis dependent is found in U.S. patent application Ser. No. 08/429,743 which is incorporated herein by reference.

Thus, it is clear that cellular proliferation, particularly endothelial cell proliferation, and most particularly angiogenesis, plays a major role in the metastasis of a cancer. If this abnormal or undesirable proliferation activity could be repressed, inhibited, or eliminated, then the tumor, although present, would not grow. In the disease state, prevention of abnormal or undesirable cellular proliferation and angiogenesis could avert the damage caused by the invasion of the new microvascular system. Therapies directed at control of the cellular proliferative processes could lead to the abrogation or mitigation of these diseases.

As described below, compounds that are useful in accordance with the invention include novel non-steroidal analogs or 2-methoxyestradiol and its derivatives that exhibit anti-mitotic, anti-angiogenic, anti-proliferative, and anti-tumor properties. Specific compounds according to the invention are described below. Preferred compounds of the invention are those derivatives of 2-methoxyestradiol (2ME₂) in which only a portion of the tetracyclic ring structure is intact. Those skilled in the art will appreciate that the invention extends to other compounds within the formulae given in the claims below, having the described characteristics. These characteristics can be determined for each test compound using the assays detailed below and elsewhere in the literature.

2-Methoxyestradiol is an endogenous metabolite of estradiol that has potent anti-proliferative activity and induces apoptosis in a wide variety of tumor and non-tumor cell lines. When administered orally, it exhibits anti-tumor and anti-proliferative activity with little or no toxicity. It is believed that the non-steroidal analogs of 2-methoxyestradiol will behave similarly. 2-Methoxyestradiol is metabolized to a less active metabolite, 2-methoxyestrone (2ME₁) as indicated by in vitro and in vivo results. Although not wishing to be bound by theory, it is believed that this metabolite is formed through the same enzymatic pathway as estrone is formed from estradiol. Although not wishing to be bound by theory, it is believed that the enzymes responsible for this reaction on estradiol are the 17β-hydroxysteroid dehydrogenases (17β-HSD) which utilize NADP+ as a co-factor (Han et al., J. Biol. Chem. 275:2, 1105-1111 (Jan. 12, 2000) and other references cited earlier). Each of the four members of this enzyme family, types 1, 2, 3, and 4, have distinct activity. It appears that 17β-HSD type 1 catalyzes the reductive reaction (estrone to estradiol), while 17β-HSD type 2 catalyzes the oxidation reaction (estradiol to estrone), and type 3 catalyzes 4-androstenedione to testosterone. It is also believed that an additional metabolic deactivation pathway results in conjugation of 2-methoxyestradiol or 2-methoxyestrone with molecules such as sulfate or glucuronic acid and subsequent loss via excretion. In this invention, non-steroidal 2-methoxyestradiol analogs and derivatives thereof may be modified to prevent these metabolic pathways from occurring.

Since 2-methoxyestradiol is metabolized to a much less active metabolite, the present invention modifies the tetracyclic ring structure (see FIG. 1) and its chemical or electrostatic characteristics for retarding or preventing interaction of the family of 17β-hydroxysteroid dehydrogenases and co-factor NADP⁺ on this substrate. This modification of chemical or electrostatic characteristics of 2-methoxyestradiol may also retard or prevent conjugation, such as glucuronidation. It is believed that retardation or prevention of these two metabolic deactivation pathways prolongs the serum lifetime of 2-methoxyestradiol and other estradiol derivatives while retaining the desired anti-angiogenic and anti-tumor activity. Assays employed for measuring glucuronidation and conjugation employ substrate enzyme uridine 5′-diphospoglucuronic acid (UDGPA).

It is well known that orally-delivered steroids such as estradiol (E₂) and ethynyl-E₂ are extensively metabolized during passage through the gastrointestinal tract and by first-pass metabolism in the liver. Two major metabolic pathways that lead to rapid deactivation and excretion are well studied (Fotsis, T.; Zhang, Y.; Pepper, M. S.; Adlercrcutz, H.; Montesano, R.; Nawreth. P. P.; Schweigerer, L., The Endogenous Estrogen Metabolite 2-Methoxyestradiol Inhibits Angiogenesis and Supresses Tumor. Nature, 1994, 368, 237-239; Wang, Z.; Yang, D.; Mohanakrishnan, A. K.; Fanwick, P. E.; Nampoothiri, P.; Hamel, E.; Cushman, M. “Synthesis of B-Ring Homologated Estradiol Analogs that Modulate Tubulin Polymerization and Microtubule Stability.” J. Med. Chem., 2000, 43, 2419-2429) e.g. oxidation at the D-ring's 17-hydroxy group of E₂ to form estrone and conjugation with sulfate and/or glucuronate at the hydroxyls of position-3 on the A-ring and position-17 on the D-ring.

Several studies have been conducted to determine SAR of 2ME₂ analogs (D'Amato, R. J.; Lin, C. M.; Flynn, E.; Folkman, J.; Hamel, E. Inhibition of Angiogenesis and Breast Cancer in Mice by the Microtubule Inhibitors 2-Methoxyestradiol and Taxol”, Cancer Res., 1997, 57, 81-86; Cushman, M.; He, M.-H.; Katzenellenbogen, J. A.; Lin, C. M.; Hamel, E. “Synthesis, Antitubulin and Antimitotic Activity, and Cytotoxicity of Analogs of 2-Methoxyestradiol, an Endogenous Mammalian Metabolite of Estradiol that Inhibits Tubulin Polymerization by Binding to the Colchicine Binding Site.” J. Med. Chem. 1995, 38, 2041-2049; and others) but none to reduce or stop its metabolic pathway. Compounds with no chain or with variable methylene chain lengths (1-4) were synthesized by replacing hydroxyl group at position-17 of D-ring of 2ME₂ to block estrone formation or glucuronation. Similarly, several analogs of 17-deoxyestrone with modification at position-2 have been synthesized to block both the glucuronation and hydrolysis of the methoxy group to the hydroxyl. For these analogs data have been presented on the synthesis and preliminary in vitro screening in human umbilical vein endothelial cells (HUVEC) and breast cancer tumor MDA-MB-231 cells for antiproliferative activity, and in MCF-7 tumor cancer cells for estrogenic activity.

Anti-Proliferative Activity In Situ

Anti-proliferative activity can be evaluated in situ by testing the ability of the new non-steroidal estradiol derivatives to inhibit the proliferation of new blood vessel cells (angiogenesis). A suitable assay is the chick embryo chorioallantoic membrane (CAM) assay described by Crum et al. Science 230:1375 (1985). See also, U.S. Pat. No. 5,001,116, hereby incorporated by reference, which describes the CAM assay. Briefly, fertilized chick embryos are removed from their shell on day 3 or 4, and a methylcellulose disc containing the drug is implanted on the chorioallantoic membrane. The embryos are examined 48 hours later and, if a clear avascular zone appears around the methylcellulose disc, the diameter of that zone is measured. Using this assay, a 100 μg disk of the estradiol derivative 2-methoxyestradiol was found to inhibit cell mitosis and the growth of new blood vessels after 48 hours. This result indicates that the anti-mitotic action of 2-methoxyestradiol can inhibit cell mitosis and angiogenesis.

Anti-Proliferative Activity In Vitro

In this invention, analogs of 2-methoxyestradiol which are non-steroidal in structure are proposed to have similar biological properties to 2-methoxyestradiol. The process by which 2ME₂ or its analogs affects cell growth remains unclear, however, a number of studies have implicated various mechanisms of action and cellular targets. 2ME₂ induced changes in the levels and activities of various proteins involved in the progression of the cell cycle. These include cofactors of DNA replication and repair, e.g., proliferating cell nuclear antigen (PCNA) (Klauber, N., Parangi, S., Flynn, E., Hamel, E. and D'Amato, R. J. (1997), Inhibition of angiogenesis and breast cancer in mice by the microtubule inhibitors 2-methoxyestradiol and Taxol., Cancer Research 57, 81-86; Lottering, M-L., de Kock, M., Viljoen, T. C., Grobler, C. J. S. and Seegers, J. C. (1996) 17β-Estradiol metabolites affect some regulators of the MCF-7 cell cycle. Cancer Letters 110, 181-186); Cell division cycle kinases and regulators, e.g., p34^{cdc2} and cyclin B (Lottering et al. (1996); Attalla, H., Mäkelä, T. P., Adlercreutz, H. and Andersson, L. C. (1996) 2-Methoxyestradiol arrests cells in mitosis without depolymerizing tubulin. Biochemical and Biophysical Research Communications 228, 467-473; Zoubine, M. N., Weston, A. P., Johnson, D. C., Campbell, D. R. and Banerjee, S. K. (1999) 2-Methoxyestradiol-induced growth suppression and lethality in estrogen-responsive MCF-7 cells may be mediated by down regulation of p34cdc2 and cyclin B1 expression. Int J Oncol 15, 639-646); transcription factor modulators, e.g., SAPK/JNK (Yue, T-L., Wang, X., Louden, C. S., Gupta, L. S., Pillarisetti, K., Gu, J-L., Hart, T. K., Lysko, P. G. and Feuerstein, G. Z. (1997) 2-Methoxyestradiol, an endogenous estrogen metabolite induces apoptosis in endothelial cells and inhibits angiogenesis: Possible role for stress-activated protein kinase signaling pathway and fas expression. Molecular Pharmacology 51, 951-962; Attalla, H., Westberg, J. A., Andersson, L. C., Aldercreutz, H. and Makela, T. P. (1998) 2-Methoxyestradiol-induced phosphorylation of bc1-2: uncoupling from JNK/SAPK activation. Biochem and Biophys Res Commun 247, 616-619); and regulators of cell arrest and apoptosis, e.g., tubulin (D'Amato, R. J., Lin, C. M., Flynn, E., Folkman, J. and Hamel, E. (1994) 2-Methoxyestradiol, and endogenous mammalian metabolite, inhibits tubulin polymerization by interacting at the colchicine site. Proc. Natl. Acad. Sci. USA 91, 3964-3968; Hamel, E., Lin, C. M., Flynn, E. and D'Amato, R. J. (1996) Interactions of 2-methoxyestradiol, and endogenous mammalian metabolite, with unploymerized tubulin and with tubulin polymers. Biochemistry 35, 1304-1310), p21^{WAF1/CIP1} (Mukhopadhyay, T. and Roth, J. A. (1997) Induction of apoptosis in human lung cancer cells after wild-type p53 activation by methoxyestradiol. Oncogene 14, 379-384), bc1-2 and FAS (Yue et al. (1997); Attalla et al. (1998)), and p53 (Kataoka, M., Schumacher, G., Cristiano, R. J., Atkinson, E. N., Roth, J. A. and Mukhopadhyay, T. (1998) An agent that increases tumor suppressor transgene product coupled with systemic transgene delivery inhibits growth of metastatic lung cancer in vivo. Cancer Res 58, 4761-4765; Mukhopadhyay et al. (1997); Seegers, J. C., Lottering, M-L., Grobler C. J. S., van Papendorp, D. H., Habbersett, R. C., Shou, Y. and Lehnert B. E. (1997) The mammalian metabolite, 2-methoxyestradiol, affects p53 levels and apoptosis induction in transformed cells but not in normal cells. J. Steroid Biochem. Molec. Biol. 62, 253-267). The effects on the level of cAMP, calmodulin activity and protein phosphorylation may also be related to each other. More recently, 2ME₂ was shown to upregulate Death Receptor 5 and caspase 8 in human endothelial and tumor cell lines (LaVallee, T. M., Zhan, X. H., Herbstritt, C. J., Williams, M. S., Hembrough, W. A., Green, S. J., and Pribluda, V. S. 2001. 2-Methoxyestradiol induces apoptosis through activation of the extrinsic pathway. (Manuscript in preparation)). Additionally, 2ME2 has been shown to interact with superoxide dismutase (SOD) 1 and SOD 2 and to inhibit their enzymatic activities (Huang, P., Feng, L., Oldham, E. A., Keating, M. J., and Plunkett, W. 2000. Superoxide dismutase as a target for the selective killing of cancer cells, Nature. 407:390-5.). All cellular targets described above are not necessarily mutually exclusive to the inhibitory effects of 2ME₂ in actively dividing cells.

The high affinity binding to SHBG has been mechanistically associated to its efficacy in a canine model of prostate cancer, in which signaling by estradiol and 5α-androstan-3α,17β-diol were inhibited by 2ME₂ (Ding, V. D., Moller, D. E., Feeney, W. P., Didolkar, V., Nakhla, A. M., Rhodes, L., Rosner, W. and Smith, R. G. (1998) Sex hormone-binding globulin mediates prostate androgen receptor action via a novel signaling pathway. Endocrinology 139, 213-218).

The more relevant mechanisms described above have been extensively discussed in Victor S. Pribluda, Theresa M. LaVallee and Shawn J. Green, 2-Methoxyestradiol: A novel endogenous chemotherapeutic and antiangiogenic in The New Angiotherapy, Tai-Ping Fan and Robert Auerbach eds., Human Press Publisher.

Assays relevant to the mechanisms of action and cell proliferation are well-known in the art. For example, anti-mitotic activity mediated by effects on tubulin polymerization activity can be evaluated by testing the ability of an estradiol derivative to inhibit tubulin polymerization and microtubule assembly in vitro. Microtubule assembly is followed in a Gilford recording spectrophotometer (model 250 or 2400S) equipped with electronic temperature controllers. A reaction mixture typically contains 1.0M monosodium glutamate (pH 6.6), 1.0 mg/ml (10 μM) tubulin, 1.0 mM MgCl₂, 4% (v/v) dimethylsulfoxide and 20-75 μM of a composition to be tested. The reaction mixtures are incubated for 15 min. at 37° C. and then chilled on ice. After addition of 10 μl 2.5 mM GTP, the reaction mixture is transferred to a cuvette at 0° C., and a baseline established. At time zero, the temperature controller of the spectrophotometer is set at 37° C. Microtubule assembly is evaluated by increased turbity at 350 nm. Alternatively, inhibition of microtubule assembly can be followed by transmission electron microscopy as described in Example 2 of U.S. Pat. Nos. 5,504,074, 5,661,143, and 5,892,069.

Other such assays include counting of cells in tissue culture plates or assessment of cell number through metabolic assays or incorporation into DNA of labeled (radiochemically, for example ³H-thymidine, or fluorescently labeled) or immuno-reactive (BrdU) nucleotides. In addition, antiangiogenic activity may be evaluated through endothelial cell migration, endothelial cell tubule formation, or vessel outgrowth in ex-vivo models such as rat aortic rings.

Indications

The invention can be used to treat any disease characterized by abnormal cell mitosis. Such diseases include, but are not limited to: abnormal stimulation of endothelial cells (e.g., atherosclerosis), solid tumors and tumor metastasis, benign tumors, for example, hemangiomas, acoustic neuromas, neurofribomas, trachomas, and pyogenic granulomas, vascular malfunctions, abnormal wound healing, inflammatory and immune disorders, Bechet's disease, gout or gouty arthritis, abnormal angiogenesis accompanying: rheumatoid arthritis, skin diseases, such as psoriasis, diabetic retinopathy, and other ocular angiogenic diseases such as retinopathy of prematurity (retrolental fibroplasic), macular degeneration, corneal graft rejection, neuroscular glaucoma, liver diseases and Oster Webber syndrome (Osler-Weber Rendu disease).

Diseases associated with corneal neovascularization that can be treated according to the present invention include but are not limited to, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, sjogrens, acne, rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, trauma, rheumatoid arthritis, systemic lupus, polyarteritis, Wegener's syndrome, sarcoidosis, scleritis, Steven-Johnson disease, pemphigoid, radial keratotomy, and corneal graph rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

Another disease which can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Another disease that can be treated according to the present invention are hemangiomas, Osler-Weber-Rendu disease, or hereditary hemorrhagic telangiectasia, solid or blood borne tumors and acquired immune deficiency syndrome.

Other diseases that can be treated according to the present invention are various metabolic disorders, such as obesity, which is typically associated with abnormal angiogenesis and abnormal proliferative activity.

In addition, the invention can be used to treat a variety of post-menopausal symptoms, osteoporosis, cardiovascular disease, Alzheimer's disease, to reduce the incidence of strokes, and as an alternative to prior estrogen replacement therapies. The compounds of the present invention can work by estrogenic and non-estrogenic biochemical pathways.

Prodrug

The present invention also relates to conjugated prodrugs and uses thereof. More particularly, the invention relates to conjugates of estradiol compounds such as 2-methoxyestradiol and functionally active analogs and derivatives thereof, to non-steroidal derivatives of 2-methoxyestradiol without the entire tetracyclic ring structure intact, and to the use of such conjugates in the prophylaxis or treatment of conditions associated with enhanced angiogenesis or accelerated cell division, such as cancer, and inflammatory conditions such as asthma and rheumatoid arthritis, metabolic disorders including obesity, and hyperproliferative skin disorders including psoriasis. The invention also relates to compositions including the prodrugs of the present invention and methods of synthesizing the prodrugs.

In one aspect, the present invention provides a conjugated prodrug of an estradiol compound, preferably of 2-methoxyestradiol or a functionally active analog or derivative thereof, conjugated to a biological activity modifying agent.

In this invention, analogs of 2-methoxyestradiol lacking portions of the four ring substructures are proposed to have similar biological activity to 2-methoxyestradiol. These analogs will have structural components of the 2-methoxyestradiol ring system (essentially they are structural fragments of 2-methoxyestradiol), but will not have the complete steroidal backbone as shown in FIG. 1. Examples of proposed analogs are presented in the FIGS. 2 and 3 above, but the compounds of the present invention are not limited to these examples.

By “functionally active” is meant that the analog or derivative of 2-methoxyestradiol has one or more of the biological activities of 2-methoxyestradiol. The biological activities of 2-methoxyestradiol include, but are not limited to: inhibition of endothelial cell proliferation; inhibition of smooth muscle cell proliferation; inhibition of tumor cell proliferation inhibition of microtubule function; inhibition of leukocyte activation. Examples of such functionally active analogs or derivatives include 2-ethoxyestradiol, 2-hydroxyestradiol and other analogs modified at the 2 position, 2-methoxyestradiol-3-methylether, 4-methoxyestradiol, and other analogs in which the B ring is expanded to a 7-numbered ring. See also WO 95/04535 and WO 01/27132 the entire disclosures of which are incorporated herein by reference.

Alternatively, the conjugated prodrug according to the present invention includes 2-methoxyestradiol or a functionally active analog or derivative thereof, conjugated to a peptide moiety.

The incorporation of an estradiol compound such as 2-methoxyestradiol or its non-steroidal analogs, into a disease-dependently activated pro-drug enables significant improvement of potency and selectivity of this anti-cancer and anti-inflammatory agent.

In addition to the compounds of the present invention, the pharmaceutical compositions of this invention may also contain, or be co-administered (simultaneously or sequentially) with, one or more pharmacological agents of value in treating one or more disease conditions referred to hereinabove. Such pharmacological agents are well-known in the art as well as being cited elsewhere in this application and in the published documents cited in this application. Others may be found in medical texts, medical journals or on the internet.

In addition, the prodrug may be incorporated into biodegradable polymers allowing for sustained release, the polymers being implanted in the vicinity of where delivery is desired, for example, at the site of a tumor. The biodegradable polymers and their use are described in detail in Brem et al., J. Neurosurg 74:441-446 (1991).

A person skilled in the art will be able by reference to standard texts, such as Remington's Pharmaceutical Sciences 17th edition, to determine how the formulations are to be made and how these may be administered.

In a further aspect of the present invention there is provided use of a conjugated prodrug according to the present invention for the preparation of a medicament for the prophylaxis or treatment of conditions associated with angiogenesis or accelerated cell division or inflammation.

In a further aspect of the present invention there is provided a pharmaceutical composition comprising a conjugated prodrug according to the present invention, together with a pharmaceutically acceptable carrier, diluent or excipient.

The pharmaceutical composition may be used for the prophylaxis or treatment of conditions associated with angiogenesis or accelerated cell division or inflammation.

In a still further aspect of the present invention there is provided a method of prophylaxis or treatment of a condition associated with angiogenesis or accelerated or increased amounts of cell division hypertrophic growth or inflammation, said method including administering to a patient in need of such prophylaxis or treatment an effective amount of a conjugated prodrug according to the present invention, as described above.

It should be understood that prophylaxis or treatment of said condition includes amelioration of said condition.

By “an effective amount” is meant a therapeutically or prophylactically effective amount. Such amounts can be readily determined by an appropriately skilled person, taking into account the condition to be treated, the route of administration and other relevant factors. Such a person will readily be able to determine a suitable dose, mode and frequency of administration.

Pharmaceutically acceptable salts of the compound of the formula may be prepared in any conventional manner for example from the free base and acid. In vivo hydrolysable esters, amides and carbamates may be prepared in any conventional manner.

Non-Steroidal Estradiol Analog Synthesis

Known compounds that are used in accordance with the invention and precursors to novel compounds according to the invention can be purchased, e.g., from Sigma Chemical Co., St. Louis, Steraloids and Research Plus. Other compounds according to the invention can be synthesized according to known methods from publicly available precursors.

The chemical synthesis of estradiol has been described (Eder, V. et al., Ber 109, 2948 (1976); Oppolzer, D. A. and Roberts, D A. Helv. Chim. Acta. 63, 1703, (1980)). The synthetic pathways used to prepare some of the derivatives of the present invention are based on modified published literature procedures for estradiol derivatives and dimethylhydrazone (Trembley et al., Bioorganic & Med. Chem. 1995 3, 505-523; Fevig et al., J. Org. Chem., 1987 52, 247-251; Gonzalez et al., Steroids 1982, 40, 171-187; Trembley et al., Synthetic Communications 1995, 25, 2483-2495; Newkome et al., J. Org. Chem. 1966,31, 677-681; Corey et al Tetrahedron Lett 1976, 3-6; Corey et al., Tetrahedron Lett, 1976, 3667-3668) and German Patent No. 2757157 (1977).

These analogs will be prepared by a number of synthetic pathways, a general reference is a Anstead review (Anstead et al Steroids, 1997, 62, 268), which is incorporated herein by reference. It is noted that the Anstead review is a general reference on the SAR of estradiol analogs and their relationship to estrogenic activities. Accordingly, this reference (and references therein) can be used as a general source for synthetic paths for the preparation of 2ME₂ analogs that correspond to the parent estradiol compound. Additionally, AB ring analogs can be prepared from a α-tetralone precursor as shown in Scheme 1. Asymmetric preparation can be accomplished by use of chiral reagents (such as chiral bases for enolate chemistry or asymmetric hydrogenation catalysts for reductions. Some A-ring analogs can be prepared by nucleophilic addition of the appropriate alkyl Grignard or lithium reagent and subsequent reduction as in Scheme 2.

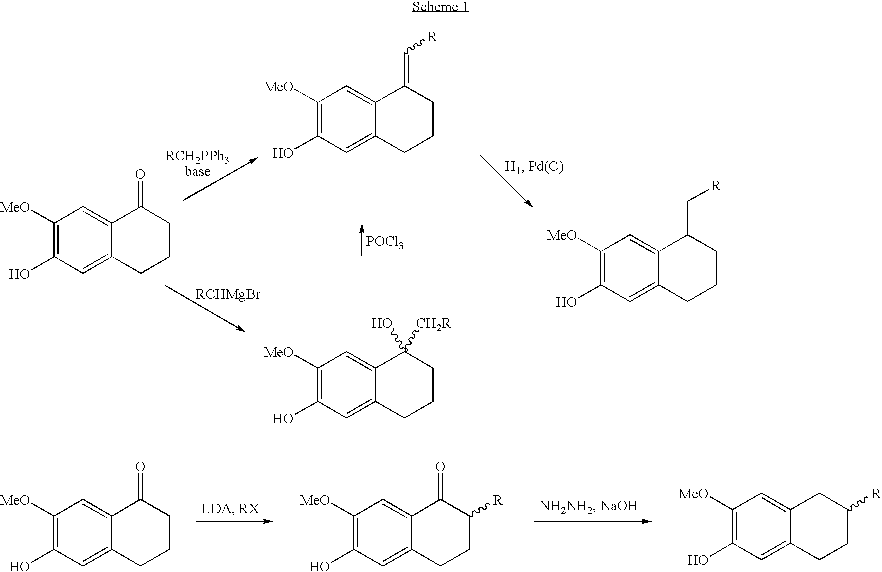

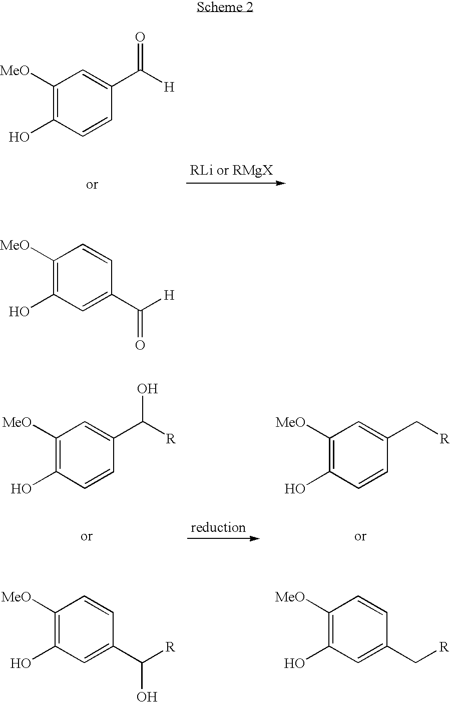

Administration

The compositions described above can be provided as physiologically acceptable formulations using known techniques, and these formulations can be administered by standard routes. In general, the combinations may be administered by the topical, oral, rectal or parenteral (e.g., intravenous, subcutaneous or intramuscular) route. In addition, the combinations may be incorporated into biodegradable polymers allowing for sustained release, the polymers being implanted in the vicinity of where delivery is desired, for example, at the site of a tumor or within or near the eye. The biodegradable polymers and their use are described in detail in Brem et al., J. Neurosurg. 74:441-446 (1991). The dosage of the composition will depend on the condition being treated, the particular derivative used, and other clinical factors such as weight and condition of the patient and the route of administration of the compound. However, for oral administration to humans, a dosage of 0.01 to 100 mg/kg/day, preferably 0.01-20 mg/kg/day, is generally sufficient.

The formulations include those suitable for oral, rectal, nasal, topical (including buccal and sublingual), vaginal or parenteral (including subcutaneous, intramuscular, intravenous, intradermal, intraocular, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tablets may be made by molding, in a suitable machine, a mixture of the powdered compound moistened with an inert liquid diluent. The tablets may optionally be coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of the present invention may include other agents conventional in the art having regard to the type of formulation in question, for example, those suitable for oral administration may include flavoring agents.

2-Methoxyestradiol is a steroidal endogenous metabolite of estradiol that has potent anti-proliferative activity and induces apoptosis in a wide variety of tumor and non-tumor cell lines. When administered orally, it exhibits anti-tumor and anti-proliferative activity with little or no toxicity. In this invention disclosure, analogs of 2-methoxyestradiol which are non-steroidal in structure are proposed to have similar biological properties to 2-methoxyestradiol.

The present invention includes compositions and methods for treating mammalian disease characterized by pathogenic angiogenesis by administering non-steroidal derivatives of 2-methoxyestradiol. In this invention, analogs of 2-methoxyestradiol lacking portions of the four ring substructures are proposed to have similar biological activity to 2-methoxyestradiol. These analogs will have structural components of the 2-methoxyestradiol ring system (essentially they are structural fragments of 2-methoxyestradiol), but will not have the complete steroidal backbone as shown above in FIG. 1. Rings that are shown in FIG. 1 as 6-member rings can also be 4, 5 or 7-member rings and may be saturated or unsaturated, and the ring shown as a five-member ring may also be a 4, 6 or 7-member ring and may be saturated or unsaturated. Examples of proposed analogs are presented in FIGS. 2 and 3, above. Although the examples illustrated in the figures are exclusively carbon chains, it is envisioned that heteroatoms, such as O, N and S may be substituted for carbon or other heteroatoms such as Si may be substituted where it is chemically possible to someone skilled in the art, without loss of the anti-angiogenic properties of these molecules. Similarly, although most of the carbon substituents are indicated as being hydrogen, some or all of these hydrogens can be replaced by more-polar moieties including but not limited to fluorines, other halides, hydroxyl, ester, amino, or alkylamine substituents which increase solubility and/or reduce metabolism and/or improve ADMET (absorption, disposition, metabolism, excretion, or toxicology) characteristics. The substituents on the unsaturated ring, which are positionally equivalent to the 2 and 3 positions of 2-methoxyestradiol and which are shown in the Figure as their preferred embodiments as methoxy and hydroxyl groups, can be replaced by groups including but not limited to halides, other alkoxy groups, propyne or other alkenes or alkynes, carboxyl or ester groups, and amines or other alkylated amino or amido groups.

In general terms, the derivatives of this invention have only a portion of the steroidal tetracyclic ring structure retained or intact. The derivatives shown in the figures above may be modified in any regiochemical position, where it is chemically possible to someone skilled in the art, at either or both the A or B rings in FIG. 2, or the A (phenyl) ring in FIG. 3. Further, the methoxy (OMe) and the hydroxy (OH) substituents shown in the structures of FIGS. 2 and 3 may also be substituted with hydrogen, as well as any C-, N-, O-, S-, P-, Si-, halogen-containing group, or other groups as indicated in the paragraphs below. Moreover, it is not necessary that these substituents be limited to the regioisomers shown, as various substitution patterns around the A and/or B rings shown in FIGS. 2 and 3 are possible, without loss of antiangiogenic and antiproliferative activity.

Combinations which are physically impossible are not contemplated by this invention, such as a carbon atom containing 5 bonds. The various substituted positions of any of the ring structures shown in FIGS. 2 and 3, and generically shown in the claims, including the methoxy and hydroxy groups of FIGS. 2 and 3, may be modified with any of the following groups:

a) alkyls (both straight and branched up to ten carbons, having either the alpha or beta stereochemistry, and may be saturated or unsaturated, substituted or unsubstituted);

b) alkenyls, including, but not limited to, olefin regio- and/or stereoisomers (E; and Z-configurations of the olefin, and the hydrocarbon chain can be straight or branched, up to ten carbons, and may be saturated or unsaturated, substituted or unsubstituted), with the C═C at any position;

c) alkynyls with either straight or branched alkyl chains, up to ten carbons; and may be saturated or unsaturated, substituted or unsubstituted, with the C≡C at any position;

d) wherein aromatic or hetero groups can be incorporated into all of the above alkyl, alkenyl and alkynyl chains either singly or in combinations thereof, and wherein the aromatic groups include but are not limited to, phenyl, phenol, aniline, anisole, toluene (ortho, meta or para derivatives), xylenes, and the hetero groups include, but are not limited to, ether, amine, carbonyl containing functional groups, alcohols, phosphates, trifluoro and thiol groups, acids, esters, sulfates, sulfonates, sulfones, sulfamates and amides;

e) mono, dialkyl or trialkyl amine substitutions with either the alpha or beta stereochemistry (alkyl can be either straight or branched, up to ten carbons);

f) —CF₂, —CHF₂, —CF₃ and longer carbon chains up to 10 carbons, such as trifluoroethanes, pentafluoroethanes, fluorinated alkyl or alkene chains up to ten carbons, with the position on the chain varying with what is chemically possible to one of skill in the art;

g) hetero groups other than those of d) and e) that are not substituted, mono-substituted or multiply substituted;

h) aromatic groups other than those of d) that are not substituted, mono-substituted or multiply-substituted;

i) both an alkyl group and a hetero or aromatic group incorporated at a single position simultaneously; and

j) geminal alkyl, hetero, or aromatic groups incorporated simultaneously (geminal is defined as two substituents at the same C).

A hetero group is defined herein as any group which contains at least one atom that is not C or H. A hetero group may contain other substituents, such as aromatic rings and other functional groups. The hetero group may be directly attached to the ring or on a substituent of a group. Especially considered are O, N, S, and P.

100% pure isomers are contemplated by this invention, however a stereochemical isomer labeled as α or β may be a mixture of both in any ratio, where it is chemically possible by one skilled in the art.

Particularly considered at substituted positions on the ring structures are the modifications of acid, amide, amine, linear and branched chain alkanes, alkenes and alkynes with heteroatom substitutions, including, but not limited to, carbonyl, —CO—, —S—, —NH—, and/or —O— instead of CH₂ and also optionally substituted with hydroxyl, amino, sulphydryl, azide, halides, nitro, azides, nitrile, sulfamate, carbamate, phosphate, azides and azos, ester, ether, halide, formamide, nitro, nitrile, sulfide, sulfoxide, sulfate, sulfamate, phosphate, and phosphonate instead of H; single or multiple homocyclic or heterocyclic rings of 3, 4, 5, 6, 7 or 8 members, either saturated or unsaturated, attached directly to the ring positions or linked via linear or branched chain alkanes, alkenes or alkynes with heteroatom substitutions, including, but not limited to, —S—, —NH—, and/or —O—, the ring hydrogens and linker hydrogens optionally being further substituted with groups, including, but not limited to those disclosed above, including, but not limited to, hydroxyl, amino, sulfhydryl and which are chemically possible for one skilled in the art.

Furthermore, at any position on the non-steroid ring structures, the following groups can be incorporated where it is chemically possible by one skilled in the art:

i) R is hydrogen;

ii) R is alkyl chains, straight and branched with stereoisomers up to 10C;

iii) R is alkene or alkyne derivatives of above alkyl chain with the olefin or alkyne moiety at any position and any configuration on the chain. Also included are multiply unsaturated alkyl chains of any configuration up to 10. The alkyl chain could be substituted with a phenyl substitutent and substituted phenyl substiutents (examples include, but are not limited to, aniline, anisole, toluene, phenol);

iv) alkyl, alkene or alkyne chains up to 10C (straight or branched) independently containing either one or multiple ester (R is defined in paragraphs ii and iii above), carboxylic acids, ketone (R is defined in paragraphs i, ii and iii above), aldehyde, alcohols, amine (primary, secondary, tertiary, and quaternary, with independent R as defined in paragraphs i, ii and iii above) nitrile, azide, urea (with R defined in paragraphs i, ii and iii above), oxime (and alkyl oxime) and halides (F, Cl, Br, I) and pharmaceutically acceptable salts of the above;

v) amines (primary, secondary, tertiary and quaternary) amines attached directly to the steroid, with R groups independently as defined in paragraphs i, ii and iii above, and pharmaceutically acceptable salts;

vi) ethers and polyethers attached directly to the steroid, where C=1 to 10;

vii) polyamines and polyols attached directly to the steroid where C=1-10;

viii) ring structures as indicated below, also including epoxides, aziridines and episulfide:

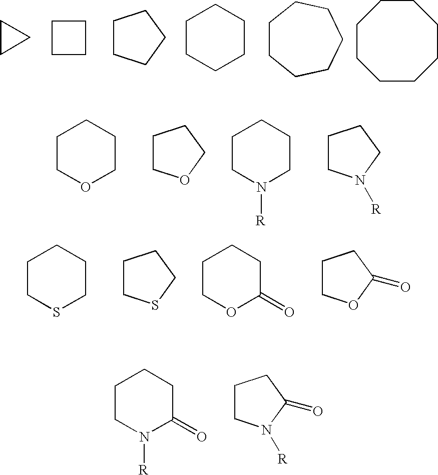

The ring structures above may have R groups (defined in parts i-vii and ix-xv) substituted at any position on the ring structure, have varying degrees of unsaturation, and be attached to any position on the steroid directly (for example, at a spiro ring junction or at a heteroatom) or through an alkyl or hetero or alkyl hetero chain, and where chemically possible to one skilled in the art;

ix) sulfate, sulfoxide, sulfamate, sulfone, sulfide, disulfide;

x) phosphate, phosphonate;

xi) nitro;

xii) amides substituted with any R group defined in paragraphs i, ii and iii above, attached to the steroid through either the carbonyl carbon or amide nitrogen, or linked to the steroid by an R group as defined in paragraphs ii and iii above;

xiii) any halogen containing alkyl, alkene and alkyne moiety (for example, CX, CX₂, CX₃ where X=F, Cl, Br, I);

xiv) —CO(CH₂)ₙOR n=0 to 10 the alkyl chain can also contain alkene or alkyne functionalities as defined in i, ii and iii above; and

xv) amino acids or peptides, naturally and unnaturally occurring, up to 20 amino acids in length.

These analogs will be prepared by a number of synthetic pathways, a general reference is a Anstead review (Anstead, et al. Steroids, 1997, 62, 268), which is incorporated herein by reference. Additionally, AB ring analogs can be prepared from a α-tetralone precursor as shown in Scheme 1 above. Asymmetric preparation can be accomplished by use of chiral reagents (such as chiral bases for enolate chemistry or asymmetric hydrogenation catalysts for reductions. Some A-ring analogs can be prepared by nucleophilic addition of the appropriate alkyl Grignard or lithium reagent and subsequent reduction as in Scheme 2 above.

These analogs and formulations will be tested in angiogenesis and anti-tumor assays both in vitro and in vivo. Several in vitro examples are HUVEC, MDA-MB-231 and MCF-7 cell proliferation assays. In vivo examples are B16 melanoma and Lewis Lung metastatic model. Other possible assays are ex vivo systems such as CAM assays and Rat Aortic Ring assays. Structure activity relationships will be examined to determine, e.g. if inversion of any stereocenter results in a change in anti-proliferative activity.

Further evaluation of these compounds can include: in vitro evaluation for antitumor, antiproliferative or antiangiogenic activity using assays such as: in vitro tumor cell line or endothelial cell proliferation assays analyzed by direct cell counts, commercial kits measuring cellular metabolic function including MTT and XTT, or cell counts using metabolic incorporation into DNA of labeled (³H-thymidine) or immunoreactive nucleotide (BrdU); in vitro assay of motility or migration including trans-membrane migration or endothelial cell layer wounding; surrogate in vitro assays for specific functions of 2ME₂ analogs such as tubulin polymerization or SOD or other enzyme binding or inhibition assays; in vitro assays for induction of apoptosis or other perturbation of cell function including TUNEL and histone analysis, oxygen radical levels, p53 levels or p53 phosphorylation, or analysis of levels or activation state of enzymes in the apoptotic pathway such as caspasesor other apoptotic molecules such as death receptors or other receptors associated with caspase activation; ex vivo assays including endothelial outgrowth from bone or aortic rings, tube forming assays, mitogenesis or motility or morphogenesis assays; or in vivo assays including chick embryo chorioallantoic membrane assay (CAM), matrigel plug assay, rabbit or mouse corneal eye pocket angiogenesis assay, liver sponge assay, or in vivo assays of angiogenesis-dependent tumor growth including B16BL6 melanoma metastasis or Lewis Lung primary and metastatic rat or mouse models or tumor xenografts or tumor development in susceptible strains such as AJ mice or mutant mouse strains such as agouti or ras-overexpressing strains or the min mouse or other transgenic or mutant mouse model systems. Examples of further analyses which can be used to determine the suitability of these analogs for use in particular diseases and pathologies include: estrogenic activity which can be assessed in vitro using estrogen dependant MCF-7 proliferation assay, or in animal assays such as uterine weight gain or uterine or vaginal cytology or diestrus time perturbation; metabolic stability which can be analyzed using liver microsomes in vitro, or dosing animals or human subjects and measuring metabolism of the compound or formation of specific metabolites such as oxidation or demethylation products or conjugates using analytical techniques including HPLC, LCMS, GCMS, or LCMSMS; models of inflammation-associated angiogenesis including psoriasis, granuloma and collagen-induced arthritis models; the ApoE −/− knockout mouse model of atherosclerotic angiogenesis; porcine model of restenosis injury; neonatal mouse model of hypoxia-driven retinopathy; measurement of cholesterol levels; assays for antiangiogenic effects on fertility or reproduction or endometriosis including inhibition of angiogenesis during follicular development; assays for effect of antiangiogenic agents on wound healing including skin punch biopsy measurement; and osteoporosis models such as in vitro measurement of osteoclast and osteoblast differentiation, proliferation, and function, ex vivo assessment of bone resorption (pitting), or in vivo measurement of bone density.

It should be understood that in addition to the ingredients, particularly mentioned above, the formulations of this invention may include other agents conventional in the art having regard to the type of formulation in question, for example, those suitable for oral administration may include flavoring agents.

In the structures, compounds, compositions, methods and descriptions provided herein, it is to be understood that: saturated bonds in any ring may be dehydrogenated where chemically possible to someone skilled in the art; all stereochemical isomers have either an α or β configuration (R and S; or D- and L-) where chemically possible to someone skilled in the art; lower alkyl is defined as a carbon chain having 1-10 carbon atoms which may be branched or unbranched and wherein chemically possible to one skilled in the art; “terminal” is defined as “at the end of a chain”; the compounds of the present invention may also be presented as a pharmaceutically acceptable salts; and examples of heterogroups that may be used include, but are not limited to, ether groups, amino groups, carbonyl groups, haloalkyl, dihaloalkyl, or trihaloalkyl groups, hydroxy groups, ester groups, dialkylamino, or monoalkylamino groups, thiol, thioether, or thioester (phosphate) groups, and oximes.

References for various syntheses, compounds, structures, compositions, methods and descriptions provided herein, include: Org. Synt. Coll. Vol. 5, 552; Org. Synt. Coll. Vol. 3, 590; and Shah, et. al. J. Med. Chem. 1995, 38, 4284; U.S. Pat. No. 5,504,074; U.S. Pat. No. 5,661,143; U.S. patent application Ser. No. 09/243,158; and U.S. patent application Ser. No. 09/939,208.

All of the publications mentioned herein are hereby incorporated by reference in their entireties. The above examples are merely demonstrative of the present invention, and are not intended to limit the scope of the appended claims.

### EXAMPLE 1

The compound shown below was prepared according to the top portion of Scheme 1, as follows.

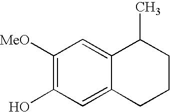

Methyl triphenylphosphonium bromide was dissolved in toluene, and t-amyl potassium alcoholate was added and the resulting mixture was refluxed for 30 min; 6-hydroxy-7-methoxy-1-tetralone was added and refluxed for 4 h. After a standard workup and purification by silica gel chromatography, a 15% yield of the olefin product (Scheme 1) was obtained. This alkene, was reduced using Pd/C (10%) and H₂ gas (at 30 psi) for 2 h, after which the reaction mixture was filtered through celite to remove the catalyst. Following column chromatography purification of the resulting filtrate, a 59% yield was obtained of the desired product shown above (mp 33.5-34.5° C.). The ¹H NMR spectrum and elemental analysis of this product were consistent with the structure shown.

We claim:

1. A compound of the general formula: 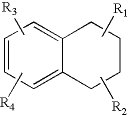
wherein R₁, R₂, R₃, and R₄ are independently selected from:
hydrogen;
a halogen;
a substituted or unsubstituted alkyl;
a substituted or unsubstituted alkenyl;
a substituted or unsubstituted alkynyl;
a substituted or unsubstituted aromatic or heterocyclic group;
a substituted or unsubstituted aralkyl;
a substituted or unsubstituted ether, amine, carbonyl containing functional group, alcohol, phosphate, trifluoro and thiol group, acid, ester, sulfate, sulfonate, sulfone, sulfamate, or amide;
a mono-, di-, or tri-substituted amine;
a cyclic or noncyclic heteroatom group;
both an alkyl group and a hetero or aromatic group incorporated at a single position simultaneously; or
geminal alkyl, hetero, or aromatic groups incorporated at a single position simultaneously.

2. A compound of the general formula: 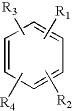
wherein R₁, R₂, R₃, and R₄ are independently selected from:
hydrogen;
a halogen;
a substituted or unsubstituted alkyl;
a substituted or unsubstituted alkenyl;
a substituted or unsubstituted alkynyl;
a substituted or unsubstituted aromatic or heterocyclic group;
a substituted or unsubstituted aralkyl;
a substituted or unsubstituted ether, amine, carbonyl containing functional group, alcohol, phosphate, trifluoro and thiol group, acid, ester, sulfate, sulfonate, sulfone, sulfamate, or amide;
a mono-, di-, or tri-substituted amine;
a cyclic or noncyclic heteroatom group;
both an alkyl group and a hetero or aromatic group incorporated at a single position simultaneously; or
geminal alkyl, hetero, or aromatic groups incorporated at a single position simultaneously.

3. A compound of the general formula: 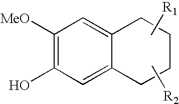
wherein R₁ and R₂ are independently selected from:
hydrogen;
a halogen;
a substituted or unsubstituted alkyl;
a substituted or unsubstituted alkenyl;
a substituted or unsubstituted alkynyl;
a substituted or unsubstituted aromatic or heterocyclic group;
a substituted or unsubstituted aralkyl;
a substituted or unsubstituted ether, amine, carbonyl containing functional group, alcohol, phosphate, trifluoro and thiol group, acid, ester, sulfate, sulfonate, sulfone, sulfamate, or amide;
a mono-, di-, or tri-substituted amine;
a noncyclic heteroatom group;
both an alkyl group and a hetero or aromatic group incorporated at a single position simultaneously; or
geminal alkyl, hetero, or aromatic groups incorporated at a single position simultaneously.

4. A compound of the general formula: 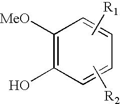
wherein R₁ and R₂ are independently selected from:
hydrogen;
a halogen;
a substituted or unsubstituted alkyl;
a substituted or unsubstituted alkenyl;
a substituted or unsubstituted alkynyl;
a substituted or unsubstituted aromatic or heterocyclic group;
a substituted or unsubstituted aralkyl;
a substituted or unsubstituted ether, amine, carbonyl containing functional group, alcohol, phosphate, trifluoro and thiol group, acid, ester, sulfate, sulfonate, sulfone, sulfamate, or amide;
a mono-, di-, or tri-substituted amine;
a cyclic or noncyclic heteroatom group;
both an alkyl group and a hetero or aromatic group incorporated at a single position simultaneously; or
geminal alkyl, hetero, or aromatic groups incorporated at a single position simultaneously.

5. A compound selected from the following group:

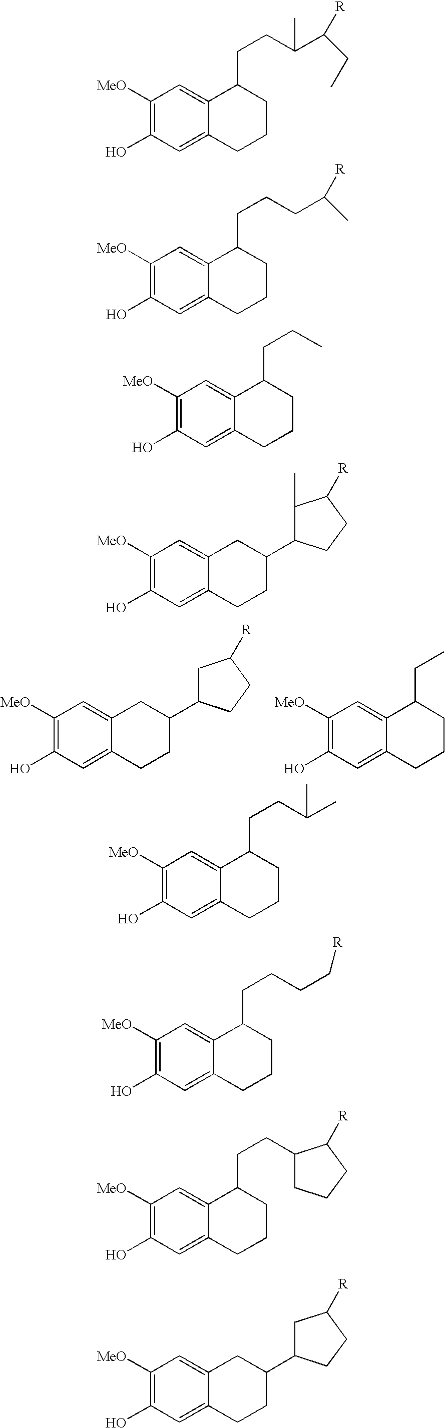

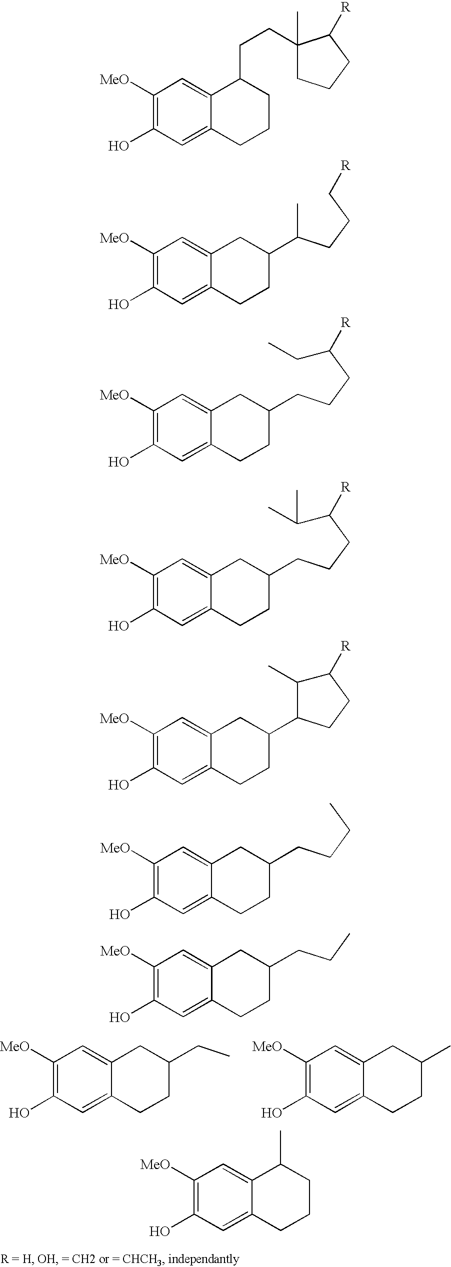

6. A compound selected from the following group:

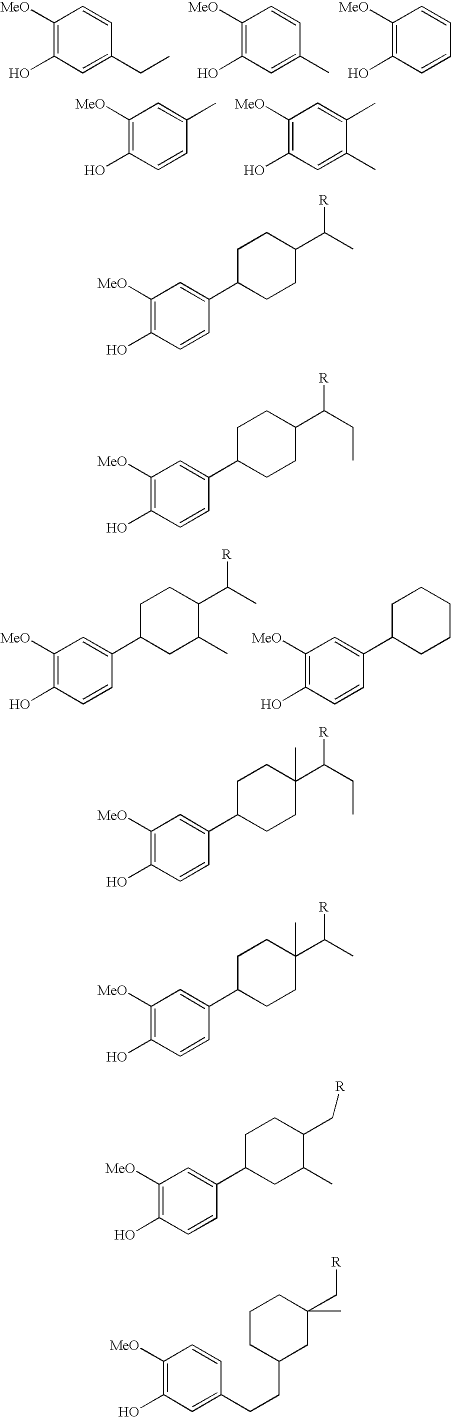

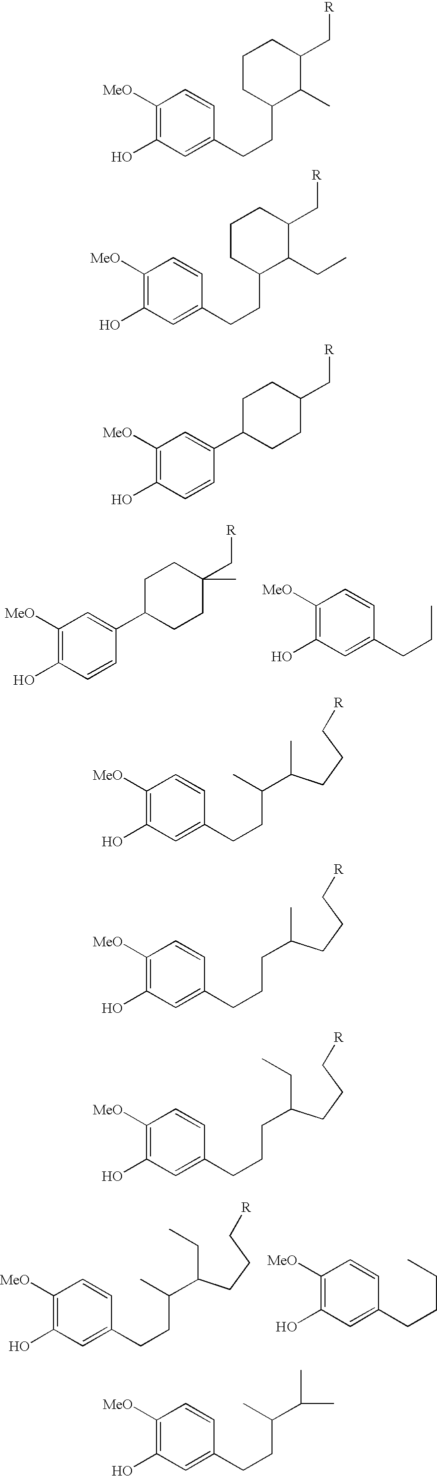

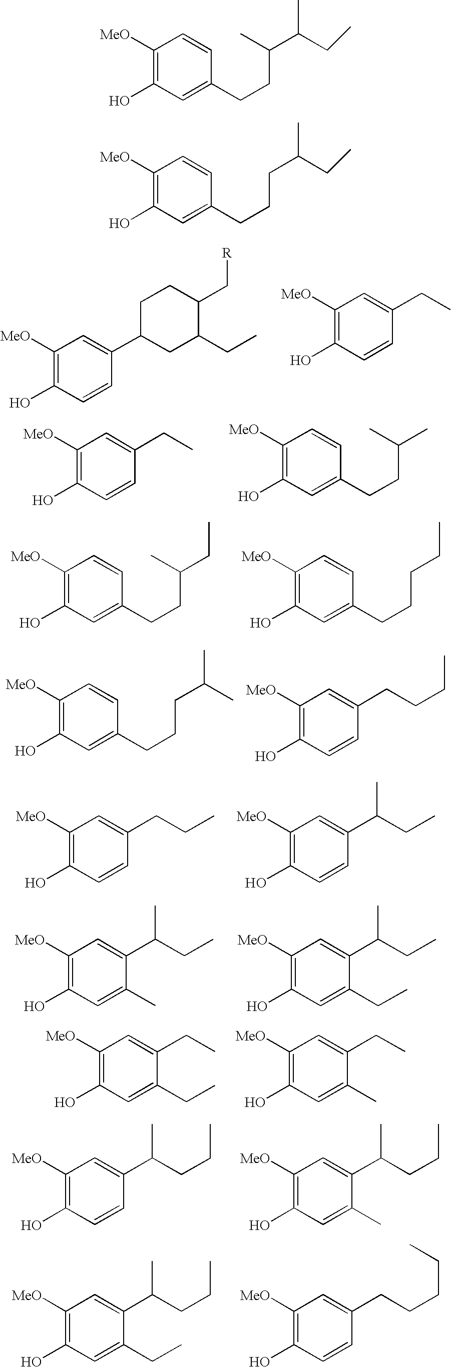

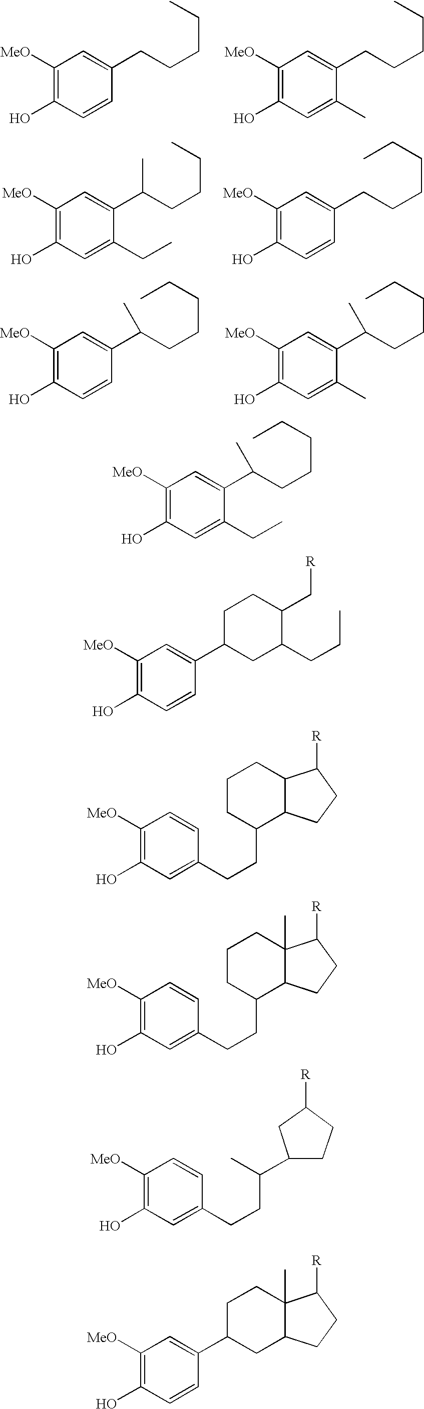

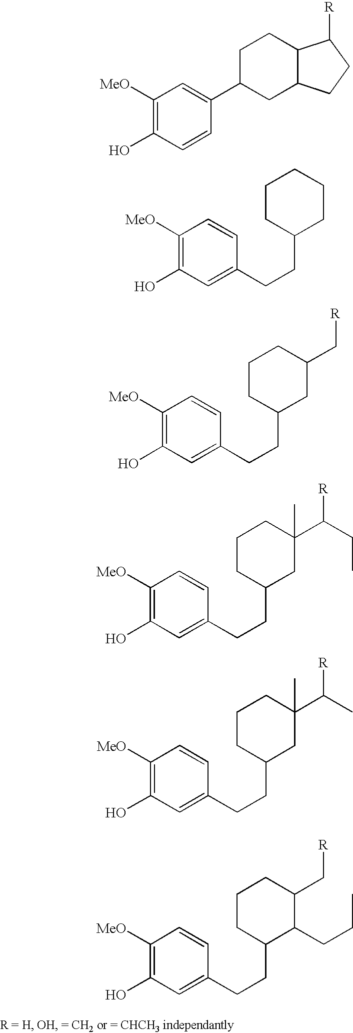

7. A method for treating a mammalian disease characterized by undesirable angiogenesis, said method comprising administering to a mammal having said undesirable angiogenesis a compound of any of claims 1 through 6, said compound being administered in an amount sufficient to inhibit angiogenesis.

8. A method for treating a mammalian disease characterized by undesirable endothelial cell proliferation, said method comprising administering to a mammal having said undesirable endothelial cell proliferation a compound of any of claims 1 through 6, said compound being administered in an amount sufficient to inhibit endothelial cell proliferation.
